(19) United States
(12) Patent Application Publication
(10) Pub. No.: US 2016/0287105 A1
(43) Pub. Date: Oct. 6, 2016

(54) CLASSIFYING A TIME-SERIES SIGNAL AS VENTRICULAR PREMATURE CONTRACTION AND VENTRICULAR TACHYCARDIA

(71) Applicant: Xerox Corporation, Norwalk, CT (US)

(72) Inventors: Luisa Fernanda POLANIA-CABRERA, Webster, NY (US); Lalit Keshav MESTHA, Fairport, NY (US)

(21) Appl. No.: 14/674,791

(22) Filed: Mar. 31, 2015

(51) Int. Cl. A61B 5/024; A61B 5/00

(52) U.S. Cl. CPC A61B 5/02416; A61B 5/7264; A61B 5/7282; A61B 5/7225

(57) ABSTRACT

What is disclosed is a system and method for classifying a time-series signal as being ventricular premature contraction, ventricular tachycardia, or normal sinus rhythm in a patient being monitored for cardiac function assessment. One embodiment hereof involves the following. A time-series signal is received which contains frequency components that relate to the function of the subject's heart. Signal segments of interest are identified in the time-series signal. Time-domain features, frequency-domain features, and non-linear cardiac dynamics are extracted from each of the identified signal segments of interest. The extracted features and dynamics become components of at least one feature vector associated with each respective signal segment of interest. Signal segments are then classified as one of: ventricular premature contraction, ventricular tachycardia, and normal sinus rhythm, based on each signal segment's respective feature vector(s).

### TECHNICAL FIELD

The present invention is directed to systems and methods for classifying a time-series signal as being ventricular premature contraction, ventricular tachycardia, or normal sinus rhythm in a patient being monitored for cardiac function assessment.

### BACKGROUND

Methods for early detection of ventricular tachycardia are increasingly needed to increase patient survival rates. Therefore, what is needed are systems and methods for classifying a time-series signal as being ventricular premature contraction, ventricular tachycardia, or normal sinus rhythm in a patient being monitored for cardiac function assessment.

### INCORPORATED REFERENCES

The following U.S. patents, U.S. patent applications, and Publications are incorporated herein in their entirety by reference.

“Classifying A Time-Series Signal As Ventricular Premature Contraction”, U.S. patent application Ser. No. 14/674,736, by Polanía-Cabrera et al., (Attorney Docket: 20141525US01 (420-P0239)).

“Method For Assessing Patient Risk For Ventricular Tachycardia”, U.S. patent application Ser. No. 14/______, by Mestha et al. (Attorney Docket: 20141576US01 (420-P0241)).

“Identifying A Type Of Cardiac Event From A Cardiac Signal Segment”, U.S. patent application Ser. No. 14/492,948, by Xu et al.

“System And Method For Detecting An Arrhythmic Cardiac Event From A Cardiac Signal”, U.S. patent application Ser. No. 14/519,607, by Kyal et al.

“Determining Cardiac Arrhythmia From A Video Of A Subject Being Monitored For Cardiac Function”, U.S. patent application Ser. No. 14/245,405, by Mestha et al.

“Discriminating Between Atrial Fibrillation And Sinus Rhythm In Physiological Signals Obtained From Video”, U.S. patent application Ser. No. 14/242,322, by Kyal et al.

“Method And Apparatus For Monitoring A Subject For Atrial Fibrillation”, U.S. patent application Ser. No. 13/937,740, by Mestha et al.

“Continuous Cardiac Signal Generation From A Video Of A Subject Being Monitored For Cardiac Function”, U.S. patent application Ser. No. 13/871,766, by Kyal et al.

“Continuous Cardiac Pulse Rate Estimation From Multi-Channel Source Video Data With Mid-Point Stitching”, U.S. patent application Ser. No. 13/871,728, by Kyal et al.

“Determining Cardiac Arrhythmia From A Video Of A Subject Being Monitored For Cardiac Function”, U.S. patent application Ser. No. 13/532,128, by Mestha et al.

“Continuous Cardiac Pulse Rate Estimation From Multi-Channel Source Video Data”, U.S. patent application Ser. No. 13/528,307, by Kyal et al.

“Estimating Cardiac Pulse Recovery From Multi-Channel Source Data Via Constrained Source Separation”, U.S. patent application Ser. No. 13/247,683, by Mestha et al.

### BRIEF SUMMARY

What is disclosed is a system and method for classifying a time-series signal as being ventricular premature contraction, ventricular tachycardia, or normal sinus rhythm in a patient being monitored for cardiac function assessment. One embodiment hereof involves the following. A time-series signal is received which contains frequency components that relate to the function of the subject's heart. Signal segments of interest are identified in the time-series signal. Time-domain features, frequency-domain features, and non-linear cardiac dynamics are extracted from each of the identified signal segments of interest. The extracted features and dynamics become components of at least one feature vector associated with each respective signal segment of interest. Signal segments are then classified as one of: ventricular premature contraction, ventricular tachycardia, and normal sinus rhythm, based on each signal segment's respective feature vector(s).

Features and advantages of the above-described method will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 is a flow diagram which illustrates one example embodiment of the present method for classifying a time-series signal for cardiac function assessment in accordance with the methods disclosed herein; and

FIG. 2 is a block diagram of one example signal processing system for performing cardiac function assessment as described with respect to the flow diagram of FIG. 1.

### DETAILED DESCRIPTION

What is disclosed is a system and method for classifying a time-series signal as being ventricular premature contraction, ventricular tachycardia, or normal sinus rhythm in a patient being monitored for cardiac function assessment.

### Non-Limiting Definitions

“Plethysmography” is the study of relative blood volume changes in blood vessels which reside beneath the surface of skin tissue.

A “photoplethysmographic (PPG) signal” is a signal obtained using an optical instrument which captures the blood volume pulse over time.

A “videoplethysmographic (VPG) signal” is a signal extracted from processing batches of image frames of a video of the skin surface.

A “subject” refers to a living being. Although the term “person” or “patient” may be used throughout this disclosure, it should be appreciated that the subject may be something other than a human such as, for example, a primate. Therefore, the use of such terms is not to be viewed as limiting the scope of the appended claims strictly to humans.

“Cardiac function” refers to the function of the heart and, to a larger extent, to the cardio-vascular system. Cardiac function can be impacted by a variety of factors including age, stress, disease, overall health, and by environmental conditions such as altitude and pressure.

“Ventricular Tachycardia” refers to an abnormal heart rate or abnormal heart rhythm. Tachycardias range from slow to fast heart rates. Some ventricular tachycardias are only slightly abnormal and have no noticeable symptoms. The teachings disclosed herein facilitate diagnosis of ventricular tachycardia.

A “time-series signal” is a signal which contains frequency components which relate to cardiac function. The time-series signal can be a photoplethysmographic (PPG) signal or a videoplethysmographic (VPG) signal. Methods for obtaining time-series signals are disclosed in several of the incorporated references by Kyal et al and Mestha et al. One or more signal segments of interest are identified in the time-series signal.

A “signal segment of interest” refers to a portion of a time-series signal which has been identified as being of interest. Methods for obtaining a segment of a signal are well established in the signal processing arts. Signal segments have a fixed length. A length of a signal segment can comprise of any of: a single cardiac cycle, a normalized cardiac cycle, multiple cardiac cycles, and multiple normalized cardiac cycles. Time-domain features, frequency-domain features and non-linear cardiac dynamics are extracted from each signal segment of interest.

“Time-domain features” refers to features obtained by analyzing peak-to-peak intervals of each signal segment of interest with respect to a mean, root mean square, and standard deviation of differences between adjacent peak-to-peak intervals and pulse amplitudes and by determining at least three features corresponding to a number of successive difference of peak-to-peak intervals which differ by more than a first time interval T1, a second time interval T2, and a third time interval T3, divided by the total number of intervals within each segment. In one embodiment, T1=25 ms, T2=15 ms, and T3=10 ms. Time-domain features become a component of a feature vector associated with a respective signal segment of interest.

“Frequency-domain features” are obtained by analyzing a signal segment of interest to determine an energy of a first and second harmonic of a fundamental frequency identified within a signal segment of interest. In another embodiment, another frequency-domain feature is the Pulse Harmonic Strength, as discussed with respect to Eq. (4). Frequency-domain features become a component of a feature vector associated with a respective signal segment of interest. The fundamental frequency and its harmonics can be identified from the power spectral density.

The “power spectral density” of a signal describes how the energy of that signal is distributed over different frequencies. In one embodiment, power P of signal x(t) is determined by averaging signal strength over a time interval [−T,T], such that:

$$P = {lim}_{T \to ∞} \frac{1}{2 T} {\int }_{- T}^{T} {x ( t )}^{2} d t$$

It is advantageous to work with a truncated Fourier transform where the signal is integrated only over a finite interval. Methods for computing the power of a given signal are well understood in the signal processing arts.

A “fundamental frequency” is the frequency of a periodic waveform with the highest energy. The fundamental frequency is given by the relationship:

$${f}_{0} = \frac{1}{T}$$

where T is the fundamental period. The first harmonic is often abbreviated as f₁. In some contexts, the fundamental f₀ is the first harmonic. If the fundamental frequency is f₀, the harmonics are given by: 2f₀, 3f₀, 4f₀, . . . , etc. Harmonics have the property that they are all periodic at the fundamental. Therefore, the sum of the harmonics is also periodic. For example, consider the two main harmonics. The energy values of the harmonics with the lowest and highest frequency of the two main harmonics are denoted as LF and HF, respectively. The ratio of these two is denoted LF/HF.

“Non-Linear Cardiac Dynamics”, also referred to herein simply as “cardiac dynamics” are extracted from each respective signal segment of interest and, in various embodiments hereof, comprise any of: Shannon Entropy as shown in Eq. (2), and a ratio as shown in Eq. (3) obtained from analyzing a Poincaré Plot.

“Shannon Entropy” is a measure of the uncertainty associated with a random variable. More specifically, it quantifies the likelihood that particular patterns exhibiting regularity over some duration of data will be followed by additional similar regular patterns over a next incremental duration of data. Higher entropy values indicate higher irregularity and complexity in time-series data. If M denotes the total number of bins, then the empirical probability distribution is calculated for each bin as:

$$p ( i ) = {N}_{bin ( i )} \text{/} {\sum }_{i = 1}^{M} {N}_{bin ( i )} \qquad ( 1 )$$

where N_{bin(i)} denotes the number of time intervals in the i^{th} bin.

Given the empirical probability distribution, the Shannon Entropy (SE) becomes:

$$SE = {\sum }_{i = 1}^{M} p ( i ) \frac{log ( p ( i ) )}{log ( \frac{1}{M} )} . \qquad ( 2 )$$

The Shannon Entropy becomes a component to a feature vector associated with a respective signal segment of interest.

“Poincaré Plot”, also referred to as “Poincaré diagram”, displays the correlation between consecutive time intervals and is constructed by plotting each peak-to-peak time interval against a next time interval. The Poincaré plot typically appears as an elongated cloud of points oriented along a line of identity. A ratio is obtained from the Poincaré plot and is given by:

SD1/SD2 (3)

where the dispersion of points along the line of identity (denoted SD1) represents the level of short-term variability, and where the dispersion of points perpendicular to the line of identity (denoted SD2) represents the level of long-term variability. The ratio of Eq. (3) becomes a component of a feature vector associated with a respective signal segment of interest.

“Pulse Harmonic Strength (PHS)” is a ratio of signal strength at the fundamental frequency and harmonics to a strength of a base signal without these fundamental frequency and harmonics. Frequencies in a neighborhood of the harmonics defines a band (e.g., 0.2 Hz or 12 beats per minutes (bpm)). All the power within this band, denoted P_{sig}, is integrated. The power in all remaining bands, denoted Pₙₒᵢ, is integrated separately. The PHS can therefore be given by the ratio:

PHS=P_{sig}/Pₙₒᵢ

Pₙₒᵢ=P_{Total}−P_{sig}. (4)

where P_{Total} is the total energy of the signal segment. The PHS represents the total strength of the pulse power because the power is centered at heart beats and the harmonics of those beats.

“Receiving a time-series signal” is intended to be widely construed and includes: retrieving, capturing, acquiring, or otherwise obtaining time-series signals for processing in accordance with the teachings hereof. Time-series signals can also be retrieved from a memory or storage device of the device used to capture those signals, or from a media such as a CDROM or DVD, retrieved from a remote device over a network, or downloaded from a web-based system or application which makes such signals available for processing.

It should be appreciated that the steps of “determining”, “analyzing”, “identifying”, “receiving”, “processing”, “classifying”, “extracting” “selecting”, “performing”, “detrending”, “filtering”, smoothing”, and the like, as used herein, include the application of any of a variety of signal processing techniques as well as mathematical operations according to any specific context or for any specific purpose. It should be appreciated that such steps may be facilitated or otherwise effectuated by a microprocessor executing machine readable program instructions such that an intended functionality can be effectively performed.

### Example Flow Diagram

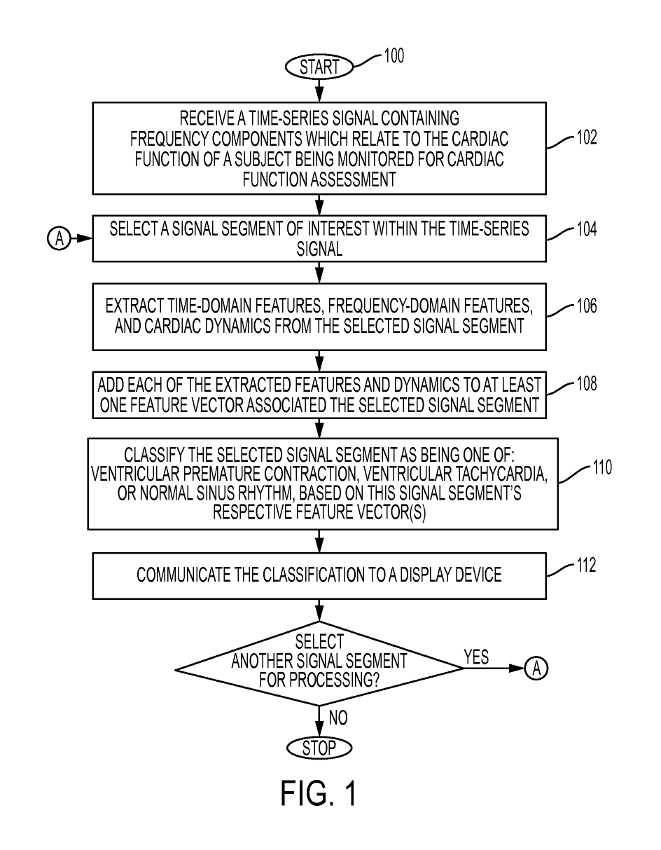

Reference is now being made to the flow diagram of FIG. 1 which illustrates one example embodiment of the present method for classifying a time-series signal for cardiac function assessment in accordance with the methods disclosed herein. Flow processing begins at step 100 and immediately proceeds to step 102.

At step 102, receive a time-series signal containing frequency components which relate to the cardiac function of a subject being monitored for cardiac function assessment.

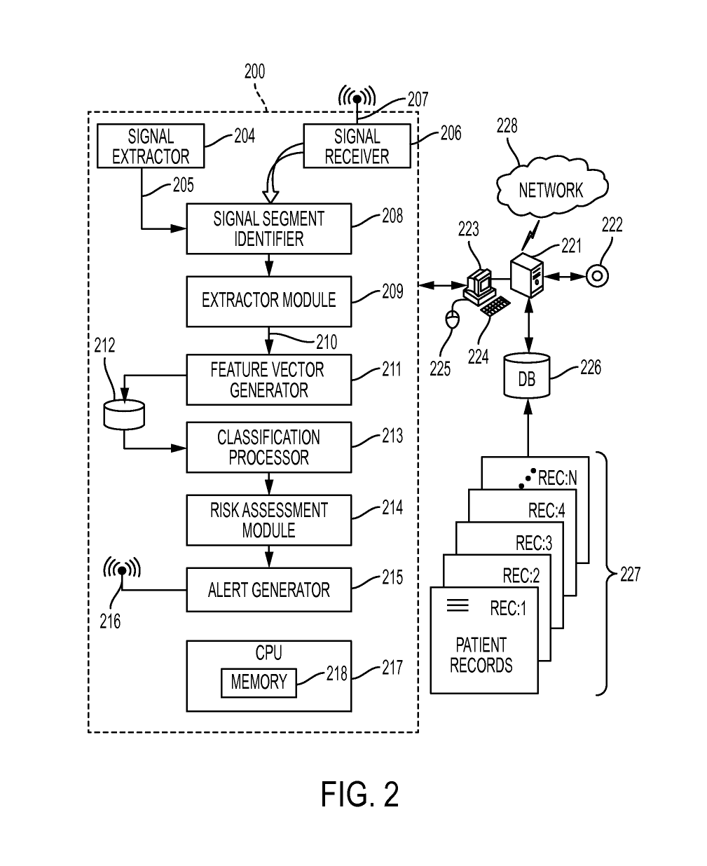

At step 104, select a signal segment of interest in the time-series signal. Signal segments have a fixed length. Such a selection may be effectuated by a user or technician using, for example, the workstation 221 of FIG. 2.

At step 106, extract time-domain features, frequency-domain features, and cardiac dynamics from the selected signal segment.

At step 108, add each of the extracted features and dynamics to at least one feature vector associated the selected signal segment. Methods for generating a vector from feature components are well understood in the mathematical arts.

At step 110, classify the selected signal segment as being one of: ventricular premature contraction, ventricular tachycardia, and normal sinus rhythm, based on this signal segment's respective feature vector(s).

At step 112, communicate the classification to a display device. One example display device is shown at 223 of FIG. 2. The classification can be communicated to a memory, a storage device, a handheld wireless device, a handheld cellular device, and/or a remote device over a network.

At step 114, a determination is made whether more signal segments remain to be classified. If not then, in this embodiment, further processing stop. Otherwise, processing repeats with respect to node B wherein, at step 104, a next signal segment is selected or is otherwise identified for processing. Processing repeats in a similar manner until no more signal segments are desired to be processed. Thereafter, further processing stops. An alert signal may be initiated in response to the classification, and a signal may be sent to a medical professional as is appropriate. Such an alert may take the form of a message displayed on a display device or a sound activated at, for example, a nurse's station or a display of a device. The alert may take the form of a colored or blinking light which provides a visible indication that an alert condition exists. The alert can be a text, audio, and/or video message. The alert signal may be communicated to one or more remote devices over a wired or wireless network. The alert may be sent directly to a handheld wireless cellular device of a medical professional. Thereafter, additional actions would be taken in response to the alert.

It should be appreciated that the flow diagrams depicted herein are illustrative. One or more of the operations in the flow diagrams may be performed in a differing order. Other operations may be added, modified, enhanced, or consolidated. Variations thereof are intended to fall within the scope of the appended claims.

### Block Diagram of Signal Processing System

Reference is now being made to FIG. 2 which illustrates a block diagram of one example signal processing system 200 for performing cardiac function assessment as described with respect to the flow diagram of FIG. 1.

Signal Extractor 204 outputs a time-series signal 205. Signal Receiver 206, in the alternative, receives a time-series signals via antenna 207. Signal Segment Identifier 208 receives the time-series signal from one or both of Signal Extractor 204 and Signal Receiver 206 and proceeds to divide the received time-series signal into signal segments of interest. The subject's cardiac specialist may facilitate such an identification of various signal segments of interest using, for instance, the display device and keyboard of the workstation 221. Once signal segments of interest have been identified or otherwise selected, Extractor Module 209 extracts time-domain features, frequency-domain features, and cardiac dynamics, as described herein, from each of the identified signal segments of interest and outputs these components (collectively at 210). The extracted features and cardiac dynamics are received by Feature Vector Generator 211 which proceeds to generate one or more feature vectors from each signal segment's respective time-domain features, frequency-domain features, and non-linear cardiac dynamics. The generated feature vectors are stored to storage device 212.

Classification Processor 213 retrieves the feature vector(s) associated with each respective signal segment from the storage device 212 and proceeds to classify each signal segment as being ventricular premature contraction, ventricular tachycardia, or normal sinus rhythm, based on each signal segment's respective feature vector(s). In one embodiment, signal segments are classified based on a magnitude of each segment's respective feature vector(s). In another embodiment, signal segments are classified using a method described in the incorporated reference entitled: “Identifying A Type Of Cardiac Event From A Cardiac Signal Segment”, by Xu et al. Other methods of classifying a signal segment based on a feature vector comprising components derived from time-domain features, frequency-domain features, and cardiac dynamics are intended to fall within the scope of the appended claims.

Risk Assessment Module 214 determines whether any of the signal segments have been classified as being ventricular premature contraction or ventricular tachycardia. If so, then module 214 signals the Alert Generator 215 to initiate an alert via antenna 216. Central Processing Unit (CPU) 217 retrieves machine readable program instructions from Memory 218 and is provided to facilitate the functionality of any of the modules of the system 200. CPU 217, operating alone or in conjunction with other processors, may be configured to assist or otherwise perform the functionality of any of the modules or processing units of the system 200, as well as facilitating communication between the system 200 and the workstation 221.

Workstation 221 has a computer case which houses various components such as a motherboard with a processor and memory, a network card, a video card, a hard drive capable of reading/writing to machine readable media 222 such as a floppy disk, optical disk, CD-ROM, DVD, magnetic tape, and the like, and other software and hardware as is needed to perform the functionality of a computer workstation. The workstation includes a display device 223, such as a CRT, LCD, or touchscreen display, for displaying information, magnitudes, feature vectors, computed values, medical information, test results, and the like, which are produced or are otherwise generated by any of the modules or processing units of the system 200. A user can view any such information and make a selection from various menu options displayed thereon. Keyboard 224 and mouse 225 effectuate a user input or selection.

It should be appreciated that the workstation 221 has an operating system and other specialized software configured to display alphanumeric values, menus, scroll bars, dials, slideable bars, pull-down options, selectable buttons, and the like, for entering, selecting, modifying, and accepting information needed for performing various aspects of the methods disclosed herein. A user may use the workstation to identify signal segments of interest, set various parameters, and facilitate the functionality of any of the modules or processing units of the system 200. A user or technician may utilize the workstation to further modify the determined magnitudes of the feature vectors as is deemed appropriate. The user may adjust various parameters being utilized or dynamically adjust, in real-time, system or settings of any device used to capture the time-series signals. User inputs and selections may be stored/retrieved in any of the storage devices 212, 222 and 226. Default settings and initial parameters can be retrieved from any of the storage devices. The alert signal initiated by Alert Generator 214 may be received and viewed on the display device 223 of the workstation and/or communicated to one or more remote devices over network 228, which may utilize a wired, wireless, or cellular communication protocol.

The workstation implements a database in storage device 226 wherein patient records are stored, manipulated, and retrieved in response to a query. Such records, in various embodiments, take the form of patient medical history stored in association with information identifying the patient (collectively at 227). It should be appreciated that database 226 may be the same as storage device 212 or, if separate devices, may contain some or all of the information contained in either storage device. Although the database is shown as an external device, the database may be internal to the workstation mounted, for example, on a hard disk therein.

Although shown as a desktop computer, it should be appreciated that the workstation can be a laptop, mainframe, tablet, notebook, smartphone, or a special purpose computer such as an ASIC, or the like. The embodiment of the workstation is illustrative and may include other functionality known in the arts. Any of the components of the workstation may be placed in communication with any of the modules of system 200 or any devices placed in communication therewith. Moreover, any of the modules of system 200 can be placed in communication with storage device 226 and/or computer readable media 222 and may store/retrieve therefrom data, variables, records, parameters, functions, and/or machine readable/executable program instructions, as needed to perform their intended functionality. Further, any of the modules or processing units of the system 200 may be placed in communication with one or more remote devices over network 228. It should be appreciated that some or all of the functionality performed by any of the modules or processing units of system 200 can be performed, in whole or in part, by the workstation. The embodiment shown is illustrative and should not be viewed as limiting the scope of the appended claims strictly to that configuration. Various modules may designate one or more components which may, in turn, comprise software and/or hardware designed to perform the intended function.

The teachings hereof can be implemented in hardware or software using any known or later developed systems, structures, devices, and/or software by those skilled in the applicable arts without undue experimentation from the functional description provided herein with a general knowledge of the relevant arts. One or more aspects of the methods described herein are intended to be incorporated in an article of manufacture. The article of manufacture may be shipped, sold, leased, or otherwise provided separately either alone or as part of a product suite or a service. The above-disclosed and other features and functions, or alternatives thereof, may be desirably combined into other different systems or applications. Presently unforeseen or unanticipated alternatives, modifications, variations, or improvements may become apparent and/or subsequently made by those skilled in this art which are also intended to be encompassed by the following claims. The teachings of any publications referenced herein are hereby incorporated by reference in their entirety.

What is claimed is:

1. A method for classifying a time-series signal as ventricular premature contraction, ventricular tachycardia, or normal sinus rhythm, in a patient being monitored for cardiac function assessment, the method comprising:
receiving a time-series signal containing frequency components which relate to a cardiac function of a subject being monitored for cardiac function assessment;
identifying at least one signal segment of interest in said time-series signal;
extracting time-domain features, frequency-domain features, and cardiac dynamics from each of said signal segments of interest;
adding each of said extracted features and cardiac dynamics to at least one feature vector associated with each respective signal segment of interest; and
classifying each of said signal segments as being one of: ventricular premature contraction, ventricular tachycardia, and normal sinus rhythm, based on each signal segment's respective feature vector.

2. The method of claim 1, wherein said time-series signal is any of: a photoplethysmographic (PPG) signal, and a videoplethysmographic (VPG) signal.

3. The method of claim 1, wherein, in advance of extracting said features and cardiac dynamics, further comprising any of:
detrending said time-series signal to remove non-stationary components;
filtering said time-series signal to remove unwanted frequencies; and
smoothing said time-series signal to remove unwanted artifacts.

4. The method of claim 1, wherein, in advance of extracting said features and cardiac dynamics, further comprising any of:
performing automatic peak detection on said signal segment to identify cardiac pulse peaks; and
filtering said signal segment to remove cardiac pulse peaks having more than at least a 20% change in consecutive peak-to-peak intervals.

5. The method of claim 1, wherein said time-domain features are obtained by analyzing peak-to-peak intervals of said signal segments of interest with respect to the mean, root mean square, and standard deviation of differences between adjacent peak-to-peak intervals and pulse amplitudes.

6. The method of claim 5, wherein said time-domain features further comprises at least three features corresponding to a number of successive difference of peak-to-peak intervals which differ by more than a first time interval T1, a second time interval T2, and a third time interval T3, divided by a total number of intervals within said signal segment of interest.

7. The method of claim 1, wherein said frequency-domain features comprises any of: an energy of a first and second harmonic of a fundamental frequency within said signal segment of interest, and a Pulse Harmonic Strength of said signal segment.

8. The method of claim 1, wherein said cardiac dynamics comprises any of: a Shannon Entropy, and a ratio obtained from a Poincaré Plot.

9. The method of claim 1, wherein said signal segments of interest are normalized to a frequency of a normalized heartbeat.

10. The method of claim 1, wherein a length of said signal segments comprises of any of: a single cardiac cycle, a normalized cardiac cycle, multiple cardiac cycles, and multiple normalized cardiac cycles.

11. The method of claim 1, further comprising any of: initiating an alert, and signaling a medical professional.

12. The method of claim 1, further comprising communicating said classification to any of: a memory, a storage device, a display device, a handheld wireless device, a handheld cellular device, and a remote device over a network.

13. A system for classifying a time-series signal as ventricular premature contraction, ventricular tachycardia, or normal sinus rhythm, in a patient being monitored for cardiac function assessment, the system comprising:
a memory; and
a processor in communication with said memory, said processor executing machine readable program instructions for performing:
receiving a time-series signal containing frequency components which relate to a cardiac function of a subject being monitored for cardiac function assessment;
identifying at least one signal segment of interest in said time-series signal;
extracting time-domain features, frequency-domain features, and cardiac dynamics from each of said signal segments of interest;
adding each of said extracted features and cardiac dynamics to at least one feature vector associated with each respective signal segment of interest; and
classifying each of said signal segments as being one of: ventricular premature contraction, ventricular tachycardia, and normal sinus rhythm, based on each signal segment's respective feature vector.

14. The system of claim 13, wherein said time-series signal is any of: a photoplethysmographic (PPG) signal, and a videoplethysmographic (VPG) signal.

15. The system of claim 13, wherein, in advance of extracting said features and cardiac dynamics, further comprising any of:
detrending said time-series signal to remove non-stationary components;
filtering said time-series signal to remove unwanted frequencies; and
smoothing said time-series signal to remove unwanted artifacts.

16. The system of claim 13, wherein, in advance of extracting said features and cardiac dynamics, further comprising any of:
performing automatic peak detection on said signal segment to identify cardiac pulse peaks; and
filtering said signal segment to remove cardiac pulse peaks having more than at least a 20% change in consecutive peak-to-peak intervals.

17. The system of claim 13, wherein said time-domain features are obtained by analyzing peak-to-peak intervals of said signal segments of interest with respect to the mean, root mean square, and standard deviation of differences between adjacent peak-to-peak intervals and pulse amplitudes.

18. The system of claim 17, wherein said time-domain features further comprises at least three features corresponding to a number of successive difference of peak-to-peak intervals which differ by more than a first time interval T1, a second time interval T2, and a third time interval T3, divided by a total number of intervals within said signal segment of interest.

19. The system of claim 13, wherein said frequency-domain feature comprises any of: an energy of a first and second harmonic of a fundamental frequency within said signal segment of interest, and a Pulse Harmonic Strength of said signal segment.

20. The system of claim 13, wherein said cardiac dynamics comprises any of: a Shannon Entropy, and a ratio obtained from a Poincaré Plot.

21. The system of claim 13, wherein said signal segments of interest are normalized to a frequency of a normalized heartbeat.

22. The system of claim 13, wherein a length of said signal segments comprises of any of: a single cardiac cycle, a normalized cardiac cycle, multiple cardiac cycles, and multiple normalized cardiac cycles.

23. The system of claim 13, further comprising any of: initiating an alert, and signaling a medical professional.

24. The system of claim 13, further comprising communicating said classification to any of: a memory, a storage device, a display device, a handheld wireless device, a handheld cellular device, and a remote device over a [symbol]
